(19) United States
(12) Patent Application Publication
(10) Pub. No.: US 2009/0326509 A1
(43) Pub. Date: Dec. 31, 2009

(54) CONTEXT AWARE MEDICAL MONITORING AND DOSAGE DELIVERY DEVICE

(76) Inventors: Philip A. Muse, Folsom, CA (US); Tim E. Hansen, Folsom, CA (US)

Correspondence Address: COOL PATENT, P.C.;c/o CPA Global, P.O. BOX 52050, MINNEAPOLIS, MN, 55402, US

(21) Appl. No.: 12/165,179

(22) Filed: Jun. 30, 2008

(51) Int. Cl. A61M 37/00

(52) U.S. Cl. 604/503; 604/66

(57) ABSTRACT

A device and method for medical monitoring and therapeutic dosage control and delivery is disclosed.

### FIELD OF INVENTION

This disclosure relates to medical devices, more specifically the disclosure relates to medical monitoring and therapeutic substance dose delivery and control.

### BRIEF DESCRIPTION OF THE DRAWINGS

FIG. 1 is a block diagram illustrating a particular embodiment of a medical monitoring device.

FIG. 2 is a block diagram illustrating a medical monitoring and dosing process.

### DETAILED DESCRIPTION

In the following detailed description, numerous specific details are set forth in order to provide a thorough understanding of claimed subject matter. However, it will be understood by those skilled in the art that claimed subject matter may be practiced without these specific details. In other instances, well-known methods, procedures, and components have not been described in detail so as not to obscure claimed subject matter.

The term ‘therapeutic substance’ is used throughout the following disclosure and is intended to refer to any substance used for treatment, control and/or prevention of any human or animal condition, such as, for instance; prevention of disease and/or injury, healing or control of disease and/or injury and/or controlling pain, and claimed subject matter is not limited in this regard. The terms ‘dose,’ ‘dosing’ and ‘dosage’ are used throughout this disclosure and are intended to refer to the amount of a therapeutic substance to be introduced into or onto the body of a human or animal subject. The term ‘delivery’ is used throughout the following disclosure and is intended to refer to introduction of a therapeutic substance into or onto a human or animal body by any of a number of routes, such as for instance; orally, intravenously, intramuscularly, intrathecally, subcutaneously, sublingually, ocularly, nasally, inhalation, cutaneously, and/or transdermally and claimed subject matter is not limited in this regard. The term ‘subject’ is used throughout the following disclosure and is intended to refer to any human or animal to be monitored by the disclosed device and method.

In the following particular embodiments a context-aware medical monitoring and therapeutic substance delivery device and method is described for adjusting a medical regimen and including dosing based at least in part on input data and/or sensed data. Such a context-aware medical monitoring and substance delivery device may be capable of adjusting delivery of any substance to be introduced into the body of a subject, such as, medication, gases, nutrients and/or liquid, and claimed subject matter is not limited in this regard. Further, in the following particular embodiments a context-aware medical monitoring and substance delivery device is described for adjusting a medication regimen, however, such a device may be capable of adjusting a variety of other schedule parameters such as a subject's exercise schedule, resting schedule, work schedule and/or meal schedule and claimed subject matter is not limited in this regard.

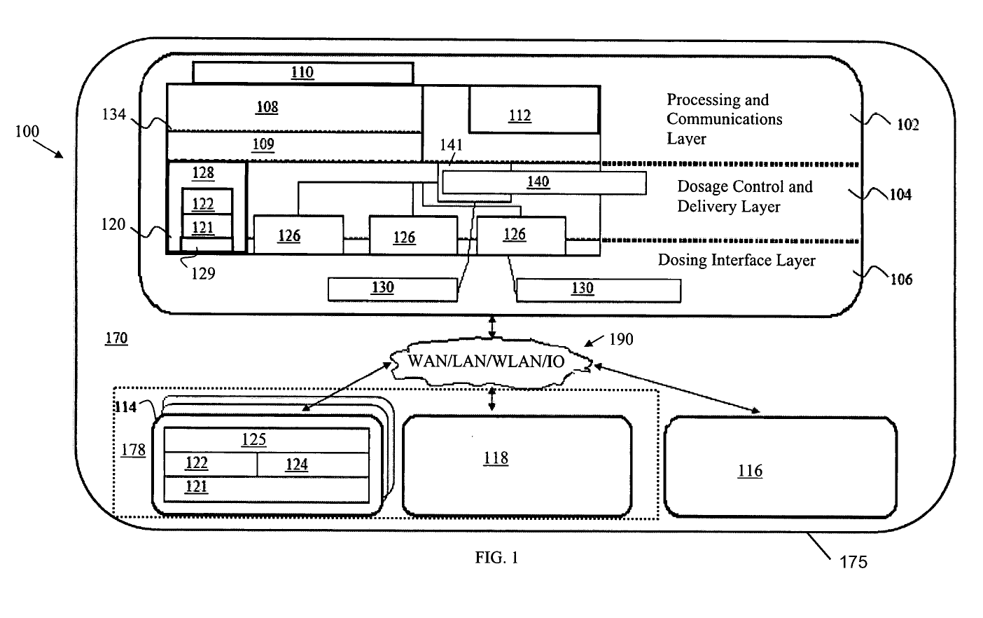

FIG. 1 is a block diagram illustrating a particular embodiment of a medical monitoring and dosing (MD) device 100 for context-aware medical monitoring and therapeutic substance delivery. According to a particular embodiment, MD device 100 may be worn by a subject, provide feedback about a subject's physiological vital signs and medical regimen, deliver therapeutic substances into and/or onto a subject's body and adjust and/or control therapeutic substance dosing delivery based on a subject's context. In a particular embodiment, MD device 100 may comprise a hardware platform 175 and software platform 170.

In a particular embodiment, hardware platform 175 may comprise processing and communications layer 102, dosing control and delivery layer 104, dosing interface layer 106 and/or distributed sensing layer 178. According to a particular embodiment, software platform 170 may comprise a computer readable media that may store programming that when executed by a processor in MD device 100 may run hardware platforms 175 and provide base services as will be described in this detailed description. However, this is merely an example of an assembly of an MD device and claimed subject matter is not so limited.

According to a particular embodiment, processing and communications layer 102 may comprise a compute and communications platform 134. In a particular embodiment, platform 134 may comprise processing unit 108 and a memory unit 109 wherein processing unit 108 may be capable of a variety of processing functions for context-aware medical monitoring and dosing control. For instance, processing unit 108 may be capable of receiving input data, sensed context data or calculated context data, combinations thereof. Processing unit 108 may further be capable of processing such data to determine a subject's physiological status, context or to determine therapeutic substance dosing schedule and amounts, or combinations thereof. In a particular embodiment, input data may be received from a variety of input devices, detected data may be received from a variety of sensors 114 and 128 and calculated context data may be received from a variety of context determining devices 118 and 129 and claimed subject matter is not limited in this regard.

In a particular embodiment, MD device 100 may communicate with a variety of other computing platforms to collaborate or provide a services. According to a particular embodiment, input data may be received via communication unit 112. Such input data may be provided by a subject and/or a third party such as a physician or caregiver. In a particular embodiment, a subject or third party may provide input data via a user terminal 110 of MD device 100 and/or via a remote communication unit 116. According to a particular embodiment, input data may be communicated via a variety of communication methods 190 including an input/output (I/O) port on MD device 100, wireline and/or wireless communication from remote communication device 116 and claimed subject matter is not limited in this regard. According to a particular embodiment, remote communications unit 116 may comprise a variety of devices such as, for instance, an onboard vehicle communication device/system, telephone, mobile phone, beeper, PC-based applications on a remote computer terminal and/or Mobile Internet Devices (MIDs) such as a PALM® device or Blackberry® and claimed subject matter is not limited in this regard.

In a particular embodiment, input data may be provided by a subject's physician and may comprise; subject baseline health, history and physical information, baseline dosing information, medication prescription, medication scheduling information, threshold parameters for medication adjustments and/or physician defined threshold parameters for context and physiology. In a particular embodiment, caregivers may provide input data such as subject response to medication (for example, medication made subject drowsy, agitated, blurred vision, etc.), subject adherence to medical regimen and/or subject status (for instance, subject is sleeping or subject is not wearing device, etc.). According to a particular embodiment, input data may comprise a subject's work, sleep, meal and/or exercise schedule. Such input data may be provided by subject manually or may be received by MD device 100 by a variety of methods such as, for instance, by synchronizing MD device 100 with a subject's electronic calendar and claimed subject matter is not limited in this regard. In a particular embodiment, baseline dose information may include such parameters as, scheduling (for example, as prescribed by physician), dose guard-bands (to allow for dosage change within physician-allowed limits) and/or patient baseline physical parameters (for example, height, weight, age and/or medical conditions). However, these are merely examples of input data that may be received by MD device 100 and claimed subject matter is not limited in this regard.

In a particular embodiment, MD device 100 may comprise local sensing platform 120 and/or a distributed sensing platform 178. In a particular embodiment, processor 108 and/or communication unit 112 may be capable of communicating with local sensing platform 120 and/or a distributed sensing platform 178. According to a particular embodiment, local sensing platform 120 may comprise one or more local sensors 128 and distributed sensing platform 178 may comprise one or more sensors 114.

In a particular embodiment, distributed sensing platform 178 and local sensing platform 120 may comprise other context calculating devices 118 or 129 which may communicate with processor 108 and/or communication unit 112. Such context calculating devices 118 and 129 may comprise a variety of devices capable of determining context related information such as position and time. Such context calculating devices may be, for example, a global positioning system receiver, a timepiece, and/or a radio frequency identification (RFID) tag and claimed subject matter is not limited in this regard. Any of sensors 114, 128 or context calculating devices 118 or 129 may be distributed via wireless communication protocols.

In a particular embodiment, sensors 114 may comprise sensor array 121, processor 122 and a communication unit 124. According to a particular embodiment, sensor 114 may also optionally comprise an onboard power source 125, such as, for instance a battery. In a particular embodiment, sensors 128 may comprise sensor array 121 and/or processor 122. In a particular embodiment, sensors 121 may detect physical phenomena and processors 122 may process sensed data and communicate the information to communication device directly or via communication unit 124. However, these are merely examples of sensor assemblies and claimed subject matter is not limited in this regard.

In a particular embodiment, sensors 114 and 128 may be capable of detecting and/or measuring a variety of physical, physiological and/or environmental phenomena such as, for instance, position, motion, acceleration, pulse, heart rate, blood pressure, blood sugar levels, body temperature, electrocardiogram (EKG) trace, percentage of oxygen in the blood (SP02), image recognition, voice/sound recognition, humidity, and/or room temperature, and claimed subject matter is not limited in this regard. In a particular embodiment, sensors 114 and 128 may comprise a variety of sensing devices such as an optical array sensor, temperature sensor, chemical sensor, chemical and/or mechanical bio-sensor, galvanic skin response sensor, camera, microphone and/or pressure sensor and claimed subject matter is not limited in this regard. According to a particular embodiment, sensor 128 may enable local sensing and sensor 114 may enable a distributed sensing system for MD device 100. However, these are merely examples of a variety of sensors that may be used in MD device 100 and claimed subject matter is not so limited.

In a particular embodiment, sensors 114 and 128 may be positioned in any variety of locations, for instance, sensor 121 may be placed on various parts of a subject's body as part of MD device 100. According to a particular embodiment, sensors 114 may be positioned in any variety of locations on various parts of a subject's body, implanted injected or ingested into a subject's body and/or positioned throughout an environment (for example, a house, car or hospital) and claimed subject matter is not limited in this regard.

In a particular embodiment, processing unit 108 may be capable of determining a variety of contexts based at least in part on analyzing input, sensed and/or calculated context data. According to a particular embodiment, processing/communication layer 102 may communicate with one or more sensors 121, 114, or devices 118 and 129 to monitor contextual factors. In a particular embodiment, in addition to determining context directly form data points (for instance, heart rate or blood sugar level), a variety of more complex contexts may be determined by MD device 100 based at least in part on analyzing input, sensed and/or calculated context data. Such complex contexts may include and are not limited to; whether a subject is adhering to a medical regimen, how a subject is responding to treatment, whether a subject is wearing MD device, whether a subject is engaging in regular vigorous activity, whether a subject is eating, working or sleeping, where a subject is located, and/or whether a subject's vital signs indicate an imminent life threatening event. Such complex contexts may be determined, in a particular embodiment, by analyzing multiple data points. For instance, multiple dimensional (3D) accelerometers may be worn on various parts of a subject's body and may enable MD device 100 to determine whether a subject is engaged in physical activity, coughing, exhibiting tremors, and/or to determine an intensity of a subject's activity level. However, these are merely examples of context determinations an MD device may make and claimed subject matter is not so limited.

In a particular embodiment, processing unit 108 may be capable of determining whether adjustments should be made to a subject's medical treatment regimen based at least in part on analyzing input, sensed and/or calculated context data. According to a particular embodiment, processor 108 may be coupled to dosage control and delivery layer 104. If processor 108 determines that an adjustment should be made to a subject's medical regimen, processor 108 may trigger a dosing adjustment mechanism in dosage control and delivery layer 104. According to a particular embodiment, processor 108 may communicate adjustments to a dosage control and delivery device controller 141 and/or may directly control various delivery devices in dosage control and delivery layer 104 and claimed subject matter is not limited in this regard. Dosage control and delivery layer 104 is discussed in greater detail further on in this disclosure.

In a particular embodiment, processor 108 may communicate a determination regarding context-aware adjustments to a subject's medical regimen to communications unit 112 and/or user interface 110. According to a particular embodiment, user interface 110 and remote communication unit 116 may be capable of communicating such information to a variety of parties, such as, a subject, caregiver and/or physician and claimed subject matter is not limited in this regard. Accordingly, processor 108 may be capable of providing information, notification and alerts to a subject and/or third party regarding a subject's medical regimen adjustments, adherence to a medical regimen and/or physiological status. For example, a dosage adjustment may be made within a range predefined by a physician and a record of dosages administered may be sent to a physician. However, if an adjustment falls outside of a predetermined range, a request for further adjustment may be routed to a physician for approval. In a particular embodiment, communication with a physician may be via communication device 116. However, this is merely an example of a dosing adjustment protocol and claimed subject matter is not limited in this regard.

According to a particular embodiment, processor 108 may additionally provide notifications to user interface 110 and/or remote communication device 116 related to a status of MD device 100. For example, MD device 100 status may be that therapeutic substance cartridge 140 needs to be replaced, delivery devices 130 need to be replaced or cleaned and/or battery power is low and claimed subject matter is not limited in this regard.

In a particular embodiment, processing unit 108 may be capable of communicating inpt, sensed and/or calculated data, analysis and dosage adjustments to a memory device 109 for storage. Such a memory device may comprise a variety of non-volatile memory devices such as flash memory and/or read only memory and claimed subject matter is not limited in this regard. Additionally, data held and communicated may be protected from unauthorized access or use by utilizing privacy and security mechanisms such as by password protection and/or encryption techniques.

In a particular embodiment, MD device 100 may comprise dosage control and delivery layer 104 capable of introducing therapeutic substances such as vitamins and pharmaceuticals into or onto the body of a subject. In a particular embodiment, dose delivery layer 104 may comprise a therapeutic substance cartridge 140 coupled to one or more dosage regulators 126 to enable fine-grained dose control of any of a variety of therapeutic substances. Dosage control and delivery layer 104 may also comprise a delivery device controller 141 for controlling delivery and dosage adjustments. However, this is merely an example of an assembly of a particular embodiment of delivery layer 104 and claimed subject matter is not limited in this regard. For instance, in another particular embodiment a dosing layer 104 may comprise multiple cartridges 140 wherein cartridges 140 comprise an onboard dose regulator and claimed subject matter is not limited in this regard.

In a particular embodiment, MD device 100 may comprise dosing interface layer 106 comprising one or more dosing devices 130. Such dosing devices 130 may comprise any of a variety of devices capable of introducing a therapeutic substance into or onto the body of a subject. For instance, such dosing devices 130 may comprise a transdermal ‘patch’ interface, a forced-air injection device, a medication injection pump, gauze (for instance, for topical applications or for fine-grain control of transdermal applications), and/or a nebulizer and claimed subject matter is not limited in this regard. Such devices 130 may be single use, or worn by a subject intermittently or continuously in order to facilitate dosing. However, these are merely examples of particular embodiments of dosing devices 130 and claimed subject matter is not limited in this regard.

In a particular embodiment, MD device 100 may be arranged such that all device components are in a single assembly, including sensor 128 as shown in FIG. 1. In another embodiment, the architecture may be distributed across different devices. For example, MD device 100 comprising distributed sensing platform 178. According to a particular embodiment, processing and communication platform 102 may collect input, sensed and/or calculated context data for aggregation in an embodiment comprising a distributed architecture. However, this is merely an example of a distributed architecture for an MD device 100 and claimed subject matter is not limited in this regard.

According to a particular embodiment, processor 108 may constantly analyze incoming data to derive context information. For example, processor 108 may be able to determine a variety of complex context, such as; whether the subject is currently wearing MD device 100, driving, participating in an activity, what sort of activity it is (for example, running, walking or sitting), when a subject is scheduled to eat, whether a subject's physiological readings are within an expected range and/or whether a subject has been adhering to a medical regimen. Based, at least in part, on a perceived context, a subject's therapeutic substance intake schedule and dosages may be altered within physician-allowed guard-bands.

In a particular embodiment, MD device 100 may make a variety of automated context-aware medication adjustments. Below are a few examples of various contexts MD device 100 may perceive and corresponding medical regimen adjustments.

In a particular embodiment, MD device 100 may detect a subject's resting heart-rate is lower than a predetermined threshold level. According to a particular embodiment, MD device 100 may lower a dosage of beta-blockers and notify physician or caregiver of the adjustment.

In a particular embodiment, MD device 100 may detect a subject's resting heart-rate is higher than a predetermined threshold level. According to a particular embodiment, MD device 100 may record history and notify subject, physician and/or caregiver.

In a particular embodiment, MD device 100 may detect a subject's blood glucose level is too high based on context. In a particular embodiment, context that may be detected are: recent exercise or other vigorous activity, meal schedule and/or work schedule based on detected context or input data such as electronic calendaring. In a particular embodiment, MD device 100 may increase a dosage of insulin and notify subject, caregiver and/or physician of the adjustment. Alternatively, adjustments to insulin dosage may be made based on electronic calendar detailing work schedule and meal schedule and claimed subject matter is not limited in this regard.

In a particular embodiment, MD device 100 may detect a subject is driving a vehicle. Such context may be detected based at least in part on external sensors in a vehicle as well as GPS data. According to a particular embodiment, MD device 100 may temporarily lower a dosage of a substance that causes drowsiness. Alternatively, MD device 100 may increase dosage of substance to enable alertness, for example Caffeine. Additionally, MD device 100 may be capable of communicating subject medical status to onboard communication systems in a vehicle, such as, On-Star® or G-Book Alpha®.

In a particular embodiment, MD device 100 may detect a subject is not wearing MD device 100. Such context may be determined via sensor 128 wherein sensor 128 is a pressure sensor or proximity sensor on MD device 100. According to a particular embodiment, MD device 100 may postpone dose delivery until subject is again wearing MD device 100. According to a particular embodiment, MD device 100 may alert subject and/or third party that MD device 100 is not being worn via a variety of automated methods such as; device alarm, automated internet phone call and/or preset text message and claimed subject matter is not limited in this regard.

In a particular embodiment, software platform 170 may comprise a computer readable media for storing programming that when executed by processor 108 may run hardware platform 175 and provide base services. In a particular embodiment, such programming may further provide higher level application capabilities, such as, for instance; wireless networking to ancillary sensing and computing platforms (for example, via Bluetooth, Institute of Electrical and Electronics Engineers (IEEE) 802.11, etc.) to exchange prescription history information as well as query a subject's calendar, sensing services to provide information to third parties about the subject, device and surrounding environment, dosage scheduling for determining when and how much to dose, dosing history to track dosage times and amounts, biotelemetry history to track physiology sensing results including dose effects on a subject, context processing to analyze sensing input (for example, subject vital signs, activity, device status, etc.) and derive context and alerting/notification function for pre-determined events (for example, subject is not wearing device during scheduled dose time or significant vital sign information). However, these are merely examples of programming applications that may run on processor 108 to provide base and higher level services in MD device 100 and claimed subject matter is not so limited.

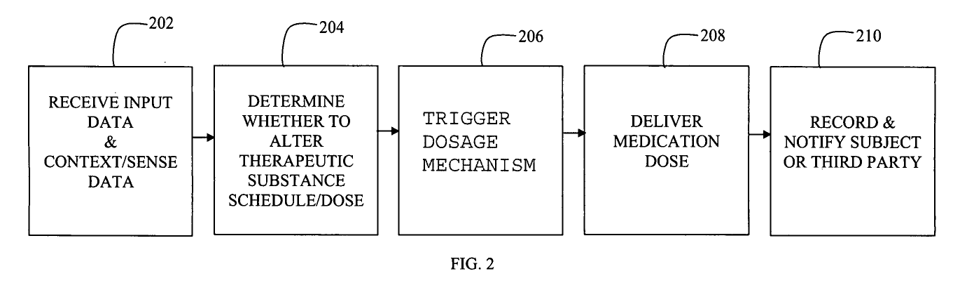

FIG. 2 illustrates a particular embodiment of a process 200 for medical monitoring and therapeutic substance dosage control and delivery. In a particular embodiment, process 200 may begin at block 202 where input, sensed and/or calculated data may be received. According to a particular embodiment, context data may comprise data related to a context of a subject that may be detected by sensors or recorded by devices capable of calculating context such as GPS devices for determining position and/or clocks for determining time of day. In a particular embodiment, input data may be any data input by a user. According to a particular embodiment, users may be a subject and/or a third party.

In a particular embodiment, process 200 may flow to block 204 where a determination whether to alter a therapeutic substance dosage or schedule and/or to send notification to a subject and/or third party may be made. Alterations to a dosage schedule or amount may be made based at least in part of analyzing input and/or context data. Notifications may be provided to a subject and/or a third party regarding subject and/or device status.

Process 200 may then flow to block 206 where a dosage delivery mechanism may be triggered by a variety of methods. For instance, dosage delivery may be triggered based on the prescribed schedule, or based on a schedule that has been altered as the result of the context of the subject. In a particular embodiment, a third party such as a physician may choose to instantaneously administer a dosage with a, “dose now” option. Such an instantaneous administration of a dose may be triggered locally or remotely via communication device 116. Further, such instantaneous dosing may be based on physiological readings, and/or based on communication directly with a subject.

In a particular embodiment, process 200 may flow to block 208 where delivery of a dosage of a therapeutic substance is executed by a variety of methods. Such methods, as discussed above, may include air-injection needle, topical application for transdermal delivery, and/or medication pump and claimed subject matter is not limited in this regard.

In a particular embodiment, process 200 may flow to block 210 where dosage delivery may be recorded and notice may be sent to a subject and/or third party. Additionally, at block 210 medical regimen adjustments, physiological readings, and other input or detected data may be stored in memory 109. However, this is merely an example of a particular embodiment of a medical monitoring and dosage delivery device and claimed subject matter is not so limited.

While certain features of claimed subject matter have been illustrated as described herein, many modifications, substitutions, changes and equivalents will now occur to those skilled in the art. It is, therefore, to be understood that the appended claims are intended to cover all such embodiments and changes as fall within the spirit of claimed subject matter.

1. An apparatus comprising:
a local sensor operable to detect context data;
a processor coupled to the sensor and operable to:
receive context data,
process the received context data to generate information associated with context data,
communicate information associated with context data;
a local communication unit coupled to the processor, wherein the local communications unit is operable to receive and communicate information associated with context data over a communications network, and receive a triggering command from the communications network; and
a dosage delivery device communicatively coupled to the processor and operable to introduce a dose of a therapeutic substance into or onto the body of a subject in response to a triggering command received from the communications network.

2. The apparatus of claim 6 wherein context data comprises input data, sensed data, detected data or calculated data, or combinations thereof.

3. The apparatus of claim 1, wherein the processor is further capable of determining an adjustment to a subject's medical regimen, the adjustment comprising an adjustment to a therapeutic substance dosage or dosing schedule, or combinations thereof based at least in part on context data.

4. The apparatus of claim 6 further comprising a dosage regulator coupled to the dosage delivery device and operable to control a therapeutic substance dosage or dosing schedule, or combinations thereof.

5. The apparatus of claim 6 wherein a therapeutic substance cartridge is coupled to the dosage delivery device and wherein the therapeutic substance cartridge is operable to control a therapeutic substance dosage or dosing schedule, or combinations thereof.

6. The apparatus of claim 3 wherein the information associated with the context data comprises, at least in part, a determination of an adjustment to be made to a subject's medical regimen.

7. The apparatus of claim 6 further comprising a memory device coupled to the processor and operable to store information associated with the context data.

8. The apparatus of claim 6 further comprising a user interface operable to receive information associated with context data.

9. The apparatus of claim 6 further comprising a remote sensor coupled to the processor and operable to detect context data.

10. The apparatus of claim 6 further comprising an onboard sensor comprising an accelerometer, optical array sensor, temperature sensor, pressure sensor, galvanic skin response sensor, camera, microphone, chemical sensor, bio-sensor, motion detector or electric impulse sensor, or combinations thereof.

11. The apparatus of claim 9 wherein the remote sensor is an accelerometer, optical array sensor, temperature sensor, pressure sensor, galvanic skin response sensor, camera, microphone, chemical sensor, bio-sensor, motion detector or electric impulse sensor, or combinations thereof.

12. The apparatus of claim 3 further comprising a context detecting device operable to detect context data and communicate context data to the processor.

13. The apparatus of claim 12 wherein the context calculating device comprises a Global Positioning System receiver, timepiece, or radio frequency identification tag, or combinations thereof.

14. The apparatus of claim 6 further comprising a remote communication unit communicatively coupled to the local communication unit wherein the remote communication unit is operable to send or receive, or combinations thereof, information associated context data over the communications network.

15. A method comprising:
receiving context data associated with a subject's context wherein the context data is associated with a subject's physiological or environmental state, or combinations thereof;
analyzing context data;
determining whether to alter a subject's therapeutic substance dosage or schedule, or combinations thereof, based at least in part on the analyzing;
triggering a dosing mechanism to deliver a therapeutic dosage into or onto the body the subject;
delivering a dosage of a therapeutic substance; and
sending notification to the subject or a third party, or combinations thereof, regarding a subject's therapeutic substance dosage status, physiological state or environmental state, or combinations thereof.

16. The apparatus of claim 6, wherein the processor is further capable of determining that the apparatus is not being worn by a subject, and
wherein the local communication unit is further capable of communicating the determination that the apparatus is not being worn by the subject over the communications network.
